(19) United States
(12) Patent Application Publication
(10) Pub. No.: US 2007/0254322 A1
(43) Pub. Date: Nov. 1, 2007

(54) Complement-based analyte assay

(76) Inventors: Alexander D. Romaschin, Toronto (CA); Christina V. Obiezu-Forster, Etobicoke (CA)

Correspondence Address: KLAUBER & JACKSON, 411 HACKENSACK AVENUE, HACKENSACK, NJ, 07601, US

(21) Appl. No.: 11/415,383

(22) Filed: May 1, 2006

(51) Int. Cl. G01N 33/53

(52) U.S. Cl. 435/7.92

(57) ABSTRACT

An assay in which the presence or quantity of analyte in a sample is determined by treating the sample to form a reagent complex in which the analyte is bound to antibody to form an immune complex that is fixed by complement. The reagent complex is captured using complement antibody, and detected using a labeled ligand for the antibody. In one embodiment, the assay is applied to detect endotoxin in patient plasma.

### FIELD OF THE INVENTION

This invention relates to assay methods and reagents, and diagnostic test kits that are useful to assay endotoxin and other analytes of interest.

### BACKGROUND OF THE INVENTION

Endotoxin, or lipopolysaccharide (LPS), is a major cell wall constituent of Gram negative bacteria, and the primary Gram negative bacterial product responsible for severe sepsis. Endotoxin is a potent trigger for the release of host pro-inflammatory mediators such as cytokines (TNF, and IL-1) from phagocytic cells, particularly monocytes, macrophages, Kupfer cells and neutrophils.

The neutrophil response to the presence of immune complex that comprises endotoxin has formed the basis of a cell-based endotoxin activity assay described by Romaschin et al, in numerous publications including U.S. Pat. No. 5,804,370; U.S. Pat. No. 6,159,683; U.S. Pat. No. 6,203,997; and U.S. Pat. No. 6,306,614; Romaschin et al, J. Immunol. Methods, 1998, 212:169-185; Romaschin et al, Sepsis, 1998, 2:119-125, Marshall et al, J. Infect. Dis., 2004, 190(3): 527-34 and see the endotoxin activity assay sold under the trade name EAA™ by Spectral Diagnostics Inc., Toronto, Canada.

In the endotoxin activity assay, whole blood comprising endotoxin and neutrophils, is reacted with a murine IgM antibody specific for the lipid A epitope of LPS to form an immune complex that activates the complement pathways and results in the fixing of complement proteins to the complement binding domains of the antibody/endotoxin complex. Neutrophils respond to the complement-fixed reagent complex via CR1 and CR3 receptor engagement which results in priming of the NADPH oxidase complex. The enhanced neutrophil oxidative burst is subsequently triggered by the addition of zymosan which results in the release of oxidants (superoxide and hydrogen peroxide) and the enzyme myeloperoxidase which generates hypochlorous acid. Through secondary reactions, and with addition of luminol, chemiluminescence provides a basis for reporting the presence of endotoxin in the original sample of whole blood. Refinements to the assay protocol provide for reliable determination of the concentration or amount of endotoxin in the sample.

The use of neutrophils and a chemiluminescent reporter for the detection of endotoxin, and other analytes of medical interest, provides a reliable and reproducible assay format. It would be desirable nevertheless to provide an alternative detection format that accommodates acellular samples such as plasma and allows for the detection of endotoxins in neutropenic patients or in stored frozen samples.

Other assay formats have been developed for the detection of immune complexes, and particularly immune complexes circulating within a patient. The detection and identification of these circulating immune complexes (CICs) provides a useful report on the immune status of the patient, and can reveal the presence of infective and other agents that elicit an immune response.

Because the CICs are routinely fixed by complement in the normal course of an immune response, and thereby present fixed complement on their surfaces, a variety of kits are available that provide complement ligands immobilized on solid phase, to capture CICs in patient samples for subsequent analysis. Different complement ligands are available in this form, including conglutinin (U.S. Pat. No. 4,740,457), anti-iC3b antibody (Quidel Corp) and a C3 binding protein produced naturally by Cuscuta europia, as described by Stanilova et al, Clin. Dev. Immunol., June-December 2003, 10(2-4):111-117.

The capture of complement-fixed ICs is applied routinely in the course of identifying the forms of immune complex that circulate in vivo. However, the applications of this technology are limited. An examination of the components of the complex can reveal only the status of the immune response in a given patient, such as the isotype of the Ig present in the complex as an indicator of the status of the immune response, or the nature of the antigen present in the complex, as an indicator of the type of immunogen responsible for the complex formation. This approach is not useful, for instance, to identify analytes that are not presented in the form of an immune complex within the circulation, such as analytes that fail to elicit an immune response, either because they are endogenous “self” analytes or because they escape immune surveillance, or overwhelm the immune system and circulate in the free form as bioavailable antigen.

It would be desirable to provide an assay useful to detect analytes of medical interest in samples of body fluid, and particularly acellular samples of human body fluid such as serum and plasma.

### SUMMARY OF THE INVENTION

The present assay is useful to detect or quantify analytes that are present in body fluid as bioavailable antigens. In the present assay, the bioavailable antigen is converted in vitro into a complement-fixed immune complex that is then detected using a labeled ligand for the immune complex.

In embodiments, the assay is applied to detect analytes present in acellular forms of body fluid, such as serum or plasma. In other embodiments, useful particularly when the sample is of human origin, the bioavailable antigen is complexed with a non-human antibody to the analyte, and labeled using antibody to the non-human antibody, thus eliminating assay background caused typically by circulating rheumatoid factor.

More particularly, and according to one aspect, the present invention provides an assay useful to determine the presence or amount of an analyte present in a sample of body fluid, the assay comprising
treating the sample to form, in vitro, a reagent complex comprising analyte present in the sample, non-human antibody to the analyte, and a complement factor fixed to the immune complex formed between said analyte and non-human antibody; and
reacting the reagent complex with a labeled ligand for said non-human antibody to the analyte,
   whereby the presence or quantity of detectable label indicates the presence or quantity of analyte in the sample.

In embodiments, the assay incorporates a solid phase comprising a ligand for the reagent complex. In preferred embodiments, the ligand binds selectively to the complement factor present in the reagent complex.

According to another aspect of the invention, there is provided a kit useful to perform an assay of the present invention, the kit comprising a first reagent in the form of a non-human antibody to an analyte of interest, and a second reagent in the form of a labeled ligand to the non-human antibody, and instructions for the use thereof to detect the presence of the analyte of interest.

In embodiments, the kit further comprises an immobilized ligand for the complement factor that becomes fixed within the reagent complex that forms during performance of the assay. In a preferred embodiment, the complement factor ligand is an antibody that, preferably, is immobilized on a solid phase such as the surface of a microwell or an inert bead.

In specific embodiments, the assay is applied for the detection or quantification of endotoxin in a sample of body fluid from a human patient, as a means for diagnosing the presence or onset of sepsis. A number of pharmaceutical companies have developed specific anti-endotoxin strategies to neutralize or inhibit the effects of endotoxin on immune effector cells. These new products include an endotoxin neutralizing lipid emulsion produced by Glaxo Smith Kline (GSK), dialysis like filters to bind and remove endotoxin from the circulation (Takeda and Toray Inc.) and a lipid A mimetic molecule which blocks the effects of endotoxin on its receptors (Eisai). Thus, in other embodiments, the present assay is useful to monitor endotoxin levels in patients undergoing sepsis therapy.

These and other aspects of the present invention are described in greater detail below with reference to the accompanying figures, in which:

### BRIEF REFERENCE TO THE DRAWINGS

FIG. 1 depicts chemiluminescence over time in whole blood or plasma with and without LPS. Dark circle with solid black line: whole blood spiked with 2.3 ng of LPS; open circle with broken line: neat whole blood; black triangle with dark solid line: EDTA-plasma spiked with 2.3 ng of LPS; open triangle with broken line: neat EDTA-plasma. RLU: relative light units.

FIG. 2 shows chemiluminescence-based signal generated with the reporter antibody, alkaline phosphatase-labeled IgM to E5, at various detector antibody concentrations;

FIG. 3 shows the impact of varying the E5 anti-LPS IgM antibody concentration on the dose response to LPS. Solid white bars denote results using 250-500 ng of E5 anti-LPS IgM; dark hatched bars denote results using 12.5-25 ng of E5 IgM.

### DETAILED DESCRIPTION OF THE INVENTION

The invention relates to an assay useful to detect analyte present in a body fluid. The assay is designed to promote the in vitro formation of a reagent complex in which analyte present in the sample becomes bound by an antibody introduced into the sample to form an analyte:antibody complex that is then fixed by complement that is either present in the sample or is added thereto in performance of the assay. Thus, formation of the reagent complex indicates the presence of analyte in the sample, and is revealed by reaction of the reagent complex with a ligand that binds the reagent complex and presents a label that can then be detected to indicate the presence of the analyte in the sample. Most desirably, the reagent complex is captured using a second, immobilized ligand for the reagent complex.

In the present assay, the analyte is any analyte that desirably (1) is bioavailable as such, as distinct from analytes and particularly foreign antigens that circulate principally, or only, as immune complexes, and (2) is or can be rendered antigenic, so that antibodies reactive with the antigen can be generated for purposes of performing the assay in accordance with the present method. It will thus be appreciated that the analyte can be a protein, peptide or polypeptide such as an antibody; a nucleic acid including DNA or RNA; a saccharide, polysaccharide or carbohydrate; a fatty acid; or a lipid, as well as combinations thereof including lipoproteins, lipopolysaccharides, glycosylated proteins, etc. The analyte can also be a hapten such as a small molecule including a drug of abuse or a pharmaceutical, as well as any other analyte of interest or concern for instance in the agricultural or environmental fields. Desirably, but not essentially, the analyte is of medical interest and is a marker useful in diagnosing a medical condition. In a specific embodiment of the present invention, the analyte is the lipopolysaccharide indicative of infection by a Gram negative microorganism, and is useful in diagnosing the onset or progression of sepsis. Alternatively, the analyte is the lipoteichoic acid characteristic of Gram positive infection. Of course, the analyte can also be a marker of infection by other organisms including a virus, such as a flavivirus including West Nile Virus and dengue virus, HIV, CMV, HepB, HepC and the like, a fungus such as yeast, Aspergillus, Trichoderma and the like, a parasite such as the malaria parasite, etc.

The analyte antibodies useful in the present invention can be any type that (1) binds to the analyte of interest, and (2) forms, with the analyte, and antibody:analyte complex that can be fixed by complement. Most desirably, the antibody is one that can trigger complement fixation in its antigen-complexed form, by a complement factor for which components of the complement cascade are resident in, i.e., endogenous to the sample. Because complement activation results in the binding of complement factor to sites in the Fc region of the complexed antibody, it will be appreciated that intact antibodies are suitable rather than Fc-truncated fragments such as Fab fragments or single chain Fv fragments. However, the useful antibodies otherwise include the Fc-bearing native or engineered antibodies that are efficient in complement activation such as mouse IgM, and certain isotypes of murine IgG, including polyclonal and monoclonal forms thereof as well as engineered forms thereof.

In a particularly preferred aspect of the present invention, the species origin of the analyte-binding antibody is different from the species origin of the sample. For example, in the case where the sample is of human origin, the antibody most desirably is not of human origin, and may be produced for instance in another other host such as mouse, rat, rabbit, goat, sheep, etc. This has the advantage of avoiding interference by rheumatoid factor or by other endogenous antigen-antibody complexes present in the sample during performance of the assay on samples of human body fluid.

The present assay can be performed on samples of various types, including cerebrospinal fluid, semen, saliva, urine and whole blood including cell-free (acellular) fractions thereof such as serum and plasma including EDTA-supplemented plasma or heparinized plasma. Most desirably, the body fluid sample comprises the complement factors required to fix complement to the antibody:analyte complexes formed during the present assay. In the alternative, but less conveniently, the complement factors can be added to the sample to provide for the required fixation of the complement factor.

In a particularly preferred embodiment, the sample of body fluid is a sample of plasma, and particularly an EDTA-supplemented sample of plasma.

The present assay thus promotes the formation of a detectable reagent complex that comprises complement factor fixed to analyte:antibody complexes. Detection of this complex can be achieved by any of a wide variety of formats used currently in the field of in vitro diagnostics. In embodiments, the complex is reacted with a detector ligand that bears a label that is either visible to the naked eye or measurable by appropriate instrumentation. The detector ligand for the complex can bind, selectively, to any component within the complex, including the complement factor, the analyte or the antibody, and regions that are conformationally unique to the complex. The detector ligand can be any agent that binds to the intended target. In the case where the ligand binds to an agent in the reagent complex that is also present in the sample, such as the analyte or the complement factor, there is desirably a capture step that allows for the removal of any unbound target. Desirably, the ligand is one that binds the antibody of the analyte that is introduced to the sample. Preferably, the ligand is an antibody to the analyte antibody. For instance, in the case where the analyte antibody is a murine IgM, the detector ligand can be an antibody to murine IgM, such as a goat antibody to murine IgM.

The detector ligand comprises a bound label that can also be any of the various types used currently in the field of in vitro diagnostics, including particulate labels including metal sols such as colloidal gold, isotopes such as I125 or Tc99 presented for instance with a peptide chelating agent, chromophores including fluorescent markers, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase and the like. In a specific embodiment, the label is the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.1^{3.7}}decan}-4-yl) phenyl phosphate (CSPD), as well as CDP and CDP-star® makreted by Tropix (Bedford, Mass.) or Lumigen PPD, Lumi-Phos, and Lumi-Plus by Lumigen Inc. (Southfield, Mich.) or other luminescent substrates well-known to those in the art for example the chelates of suitable lanthanides such as Terbium(III) and Europium(III).

The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice. In a specific embodiment, the label is alkaline phosphatase, and the detection thereof is achieved using an alkaline phosphatase substrate such as CDP to generate a signal detectable as chemiluminescence, using for instance a chemiluminometer such as the Autolumat LB953 available from E.G.&G.Berthold in Wildbad, Germany.

Conveniently, the present assay incorporates a solid phase to facilitate removal of contaminating agents that are not bound during formation of the reagent complex. The solid phase comprises a capture ligand that binds selectively to the reagent complex, at a site distinct from the binding site for the detector ligand, to allow formation of a so-called sandwich in which the reagent complex is bound simultaneously by the capture ligand and the detector ligand. The capture ligand is desirably immobilized through binding to a solid phase that can for instance include a glass or plastic surface such as a slide or microwell, or a bead such as a bead formed from latex or other polymer. The bead can further be magnetizable, so that separation of the bead and bound reagents from other components can be achieved conveniently by applying a magnetic field. In embodiments, the capture ligand is immobilized by conjugation to a magnetic bead, such as paramagnetic beads, such as the beads available from Dynal.

Thus, in specific aspects, the present invention provides an assay useful to determine the presence or amount of an analyte, the assay comprising:
Obtaining a sample of a body fluid suspected of containing said analyte, said sample comprising endogenous complement factors capable of fixing complement,
Adding, to the sample, a non-human antibody to said analyte, thereby to form in vitro a reagent complex comprising an analyte:non-human antibody complex having a complement factor fixed thereto;
reacting the reagent complex with a detector ligand comprising ligand for said non-human antibody and a detectable label; and
determining the presence or quantity of label bound to said reagent complex, thereby to determine the presence or quantity of analyte in said sample.

In embodiments, the assay further comprises the step of capturing the reagent complex, preferably before or alternatively after reaction with the detector ligand, with an immobilized capture ligand, wherein the capture ligand binds selectively to the complement factor fixed therein. Suitable capture ligands include, as noted above, conglutinin and C3 binding proteins produced naturally by Cuscuta europia. In a preferred embodiment, the capture ligand is an antibody to said complement factor. In other embodiments, the capture ligand is conjugated to a bead, and preferably a magnetizable bead. In other embodiments, the capture ligand is antibody to a fixed C3 breakdown product such as C3b, C3d or preferably iC3b, wherein the antibody is conjugated to a magnetic or magnetizable bead.

In general, the activation of complement by immune complexes of IgM and IgG involves the classical complement pathway and the formation of the C3 convertase C4b2b. This C3 convertase is formed when a conformational change occurs in the antibody upon binding of the specific antigen. The complement factor C1q binds at least 2 conformationally altered immunoglobulin Fc domains and activates a protease cascade resulting in C1s cutting peptides from C2 and C4. This proteolytic action exposes an active thioester bond on the cleaved C4 (C4b) which covalently binds to the antigen surface. The covalently bound C4b combines with proteolytically cleaved C2 (C2b) in a non-covalent complex to form the classical pathway C3 convertase enzyme which is bound to the antigen. This convertase then acts as an enzyme to cleave intact C3 into C3b which covalently deposits on binding sites on the antigen. This process results in the immune complex becoming opsonized and labeled with complement molecules for which there are specific receptors. The C3b bound to the antigen can be further proteolytically cleaved to form iC3b by complement regulatory proteins factors H and I. Cell receptors (CR1) for C3b are found in high concentrations on erythrocytes and also neutrophils. Receptors for iC3b are also present in neutrophils and other immunocompetent cells. In concert these receptors facilitate the clearance and disaggregation of immune complexes to prevent their deposition in tissues such as kidney. Therefore, the detection of in vitro generated immune complexes as described herein involves the recognition of either C3b or iC3b or both molecules deposited on immune complexes.

Thus, to assay samples which do not contain complement proteins it is appropriate to add frozen plasma or recombinant complement proteins minimally composed of C1, C2, C3 and C4 in order to provide sufficient complement proteins to support antibody: antigen opsonization, and to allow for capture based on fixed C3b or iC3b.

In a specific embodiment of the present invention, the assay is applied for the detection of endotoxin, known also as LPS. In one embodiment of the present invention, there is provided an endotoxin detection method, comprising the steps of
Obtaining a sample of plasma or serum suspected of containing endotoxin, said sample comprising endogenous complement factors capable of fixing complement,
Adding, to the sample, a non-human antibody to said endotoxin, thereby to form in vitro a reagent complex comprising an endotoxin:non-human endotoxin antibody complex having a complement factor fixed thereto;
reacting the reagent complex with labeled antibody to said non-human antibody; and
determining the presence or quantity of label bound to said reagent complex, thereby to determine the presence or quantity of endotoxin analyte in said sample.

In embodiments, the non-human antibody is a murine antibody, such as a murine IgM. In a specific embodiment, the non-human endotoxin antibody is an antibody that binds to the lipid A component of Gram negative LPS. In a preferred embodiment, the antibody is the E5 antibody (also designated XMMEN-OE5), described for instance in U.S. Pat. No. 4,918,163, and produced by hybridoma ATCC Accession No. HB9081.

In other embodiments, the labeled antibody is an anti-murine IgM antibody. In embodiments, this antibody is labeled with alkaline phosphatase. In specific embodiment, the labeled antibody is an alkaline phosphatase-labeled anti-murine IgM antibody, such as is available from Zymed or Sigma. In other embodiments, the alkaline phosphatase label is used with a substrate that is CDP-Star/CSPD, as available from Tropix (Bedford, Mass.).

In other embodiments, the sample of body fluid is a sample of plasma prepared from whole blood extracted for instance by phlebotomy. In a specific embodiment, the sample is EDTA plasma or heparinized plasma, obtained in accordance with established procedures.

The assay is useful, as noted, to identify or quantify numerous analytes other than endotoxin. The table below illustrates embodiments thereof, and indicates the reagents useful in the performance of the assay:

TABLE

| Analyte | Antibody | Complement | Capture | Detector |
|---|---|---|---|---|
| Endotoxin | Murine anti- | C3b/iC3b | Anti-C3b or | Anti-murine |
| | LPS IgM | | iC3b or both | IgM |
| Lipoteichoic | Murine anti- | iC3b/C3b | Anti-iC3b or | Anti-murine |
| acid | LTA IgG | | iC3b or both | IgG |
| HepB Sur- | Murine anti- | C3b/iC3b | Anti-C3b or | Anti-murine |
| face Ag | HepB S antigen | | iC3b or both | IgG |
| | IgG | | | |
| Troponin | Murine anti- | C3b/iC3b | Anti-C3b or | Anti-murine |
| I | TNI IgG | | iC3b or both | IgG |
| WNV E | Murine anti- | C3b/iC3b | Anti-C3b or | Anti-murine |
| protein | WNV E protein | | iC3b or both | IgM |
| | IgM | | | |

The assay is performed generally using physiologically tolerable vehicles, buffers and temperatures. Positive and negative controls can be run in parallel, using analyte-free sample as a negative control, and analyte-spiked sample as a positive control. The quantity of analyte present in the sample can be deduced from standard curves established for a preparation spiked with a known amount of analyte. A negative control reaction that includes only the plasma/serum in conjunction with anti-IgM-ALP as well as a positive control where 2-5 ng of LPS is spiked into the plasma/serum sample can also be run in parallel. Alternatively, supplied QC controls may include a LPS-free and LPS-containing plasma/serum.

For use in performing the assay, the present invention further provides a kit comprising one or more of the required assay reagents and instructions for the use thereof in accordance with the present assay method. Many reagents useful in the assay can be purchased from other commercial sources, and it is therefore not necessary to provide these within the kit. In one embodiment, the kit comprises the non-human antibody to the target analyte. In other embodiments, the kit further comprises one or both of the immobilized capture ligand and labeled detector ligand. Optionally, the kit may further comprise the substrates useful to detect the label, for instance where the label is an enzyme requiring a chromogenic substrate for its detection. The kit may further comprise control reagents, such as a known amount of analyte. The reagents typically will be provided in either lyophilized form, with instruction for their reconstitution, or as stabilized liquid formulations, present in a suitable receptacle such as a vial, cuvette. The capture ligand may be provided either as such, or in a form already conjugated to the solid phase, such as beads or a microwell tray or the like.

In one embodiment, the endotoxin assay described herein is enabled by providing a kit comprising the murine anti-LPS IgM known as E5, magnetic beads having the capture ligand attached thereto as anti-iC3b antibody, and the detector ligand anti-murine IgM with conjugated alkaline phosphatase, optionally together with the CDP substrate. The kit may also provide a known amount of LPS, as a control reagent and may also contain LPS negative and positive plasma/serum. Alternatively, the kit may comprise, as reagents, only the murine anti-LPS IgM together with instructions for use thereof in combination with other reagents that are available commercially, as more fully described in the examples that follow.

### EXAMPLES

Methods

Bead conjugation. Anti-iC3b and anti-C3b alpha antibodies were conjugated to tosylactivated paramagnetic beads (Dynabeads, Invitrogen) according to manufacturer's instructions. Briefly, 20 μg of antibody per 1 mg of beads were incubated overnight in 0.1 M borate buffer pH 9.5 either for 48 hrs at room temperature or for 16 hrs at 37° C. Bead surface were subsequently saturated with 0.1 M borate buffer pH 9.5 supplemented with 1% purified casein of I-Block™ reagent (Tropix) for 2 hours at 37° C. followed by 2 washes with 1 mL of Tris buffered saline (TBS) pH 7.4 supplemented with 0.1% casein at 4° C. Beads were then incubated for 4 hrs of in 0.2 M Tris buffer pH 8.5 supplemented with 0.1% casein to block remaining tosyl groups. Beads were then washed as above and stored in 0.2 M Tris buffer pH 8.5 supplemented with 0.1% casein and 0.02% sodium azide.

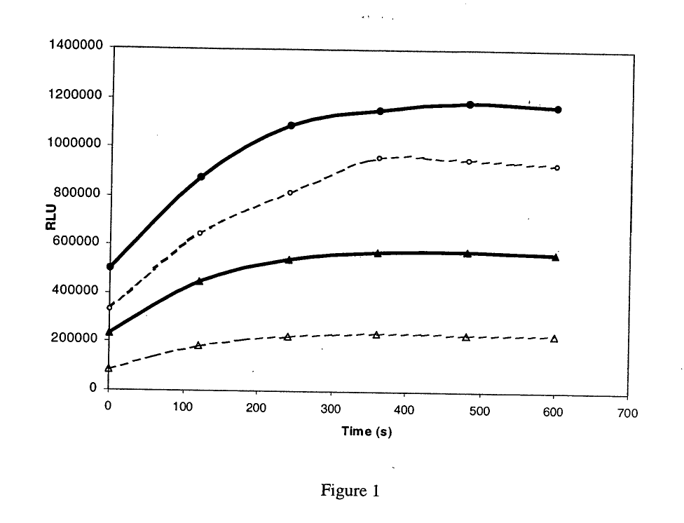

Initial assay using anti-iC3b antibody coated beads. 500 μl of plasma or whole blood samples were transferred into EAA Aliquot tubes (Spectral Diagnostics Inc.) and EAA LPSMax tube containing 2.3 ng of LPS (Spectral Diagnostics Inc.). Tubes were incubated for 10 minutes in a 37° C. heat block. Blood/plasma preparations were transferred into EAA Tubes (Tube 3) containing 1-2 μg of anti-LPS antibody and were incubated for a further 30 min in a 37° C. heat block. In the meantime, anti-iC3b antibody-conjugated beads (3 μl per reaction) were washed twice in Tris buffered saline pH 7.6 supplemented with 0.05% Tween-20 (TBS-T) and recovered on an MPC-6 magnetic platform (Invitrogen). Washed beads were then incubated with 500 μl of whole blood/plasma preparations from above with end-over-end rotation for 1 hr at 37° C. Following 3 rounds of washes with TBS-T, rabbit anti-mouse IgM-ALP (Zymed) were diluted 1 in 5000 in ELISA-Light blocking buffer (Tropix) and 100 μl was applied to beads. Beads were then incubated for 1 hr at 37° C. in pyrogen-free microtitre plate wells (Cape Cod Inc.). Beads were washed in microtitre plate wells 4 times with TBS-T, and resuspended in 200 μl of TBS-T to be transferred into Aliquot tubes (Spectral Diagnostics Inc.). Tubes were placed on magnet and TBS-T was exchanged for 150 μl of Tris pH 9.5. Chemiluminescence was monitored for 10 min in an AutoLumat LB953 following addition of 150 μl of CDP-Star EmeraldII (Tropix). Representative results of this procedure are depicted in FIG. 1.

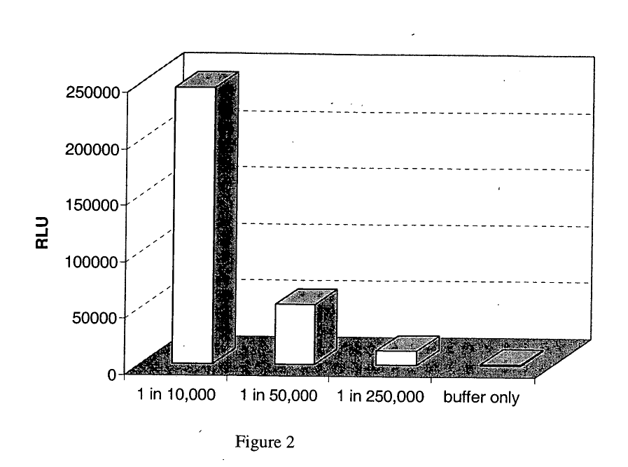

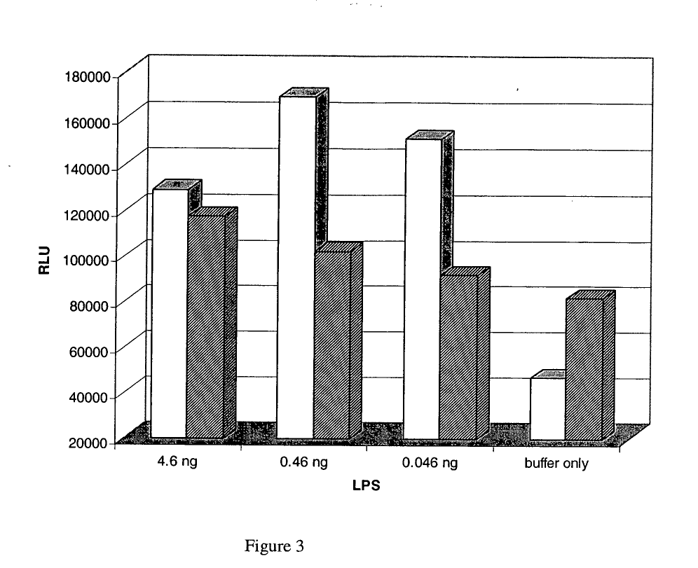

Initial assay using anti-iC3b antibody coated microtitre plates. Anti-iC3b antibody coated plates were purchased from Quidel Inc and were used to optimize assay parameters. In this procedure, anti-iC3b antibody coated microtitre plates were blocked with 100 μl of ELISA-Light blocking buffer for 3 hrs at room temperature. EAA LPSMax tubes containing various amounts of LPS as well as EAA T3 tubes containing various amounts of anti-LPS IgM were resuspended in 0.5 mL of complement activation (CA) buffer (1 mM MgCl₂, 0.15 mM CaCl₂ in TBS pH 7.6). 100 μl of LPSMax tube contents were mixed with EAA T3 contents and were incubated at 37° C. in a heat block for 10 minutes. Next, 20 μl of EDTA plasma was added to the reactions and incubation was continued for 30 minutes in a 37° C. heat block. At the end of the incubation period, 100 μl of the reactions were transferred into the microtitre plates described above and incubated with shaking for 1 hour at room temperature. Microplate wells were then washed 5 times with TBS-T as per manufacturer's instructions. One hundred μl of anti-IgM antibody conjugated to alkaline phosphatase (Zymed), diluted 1 in 20,000 in ELISA-Light blocking buffer, was added to each reaction well and incubated with shaking for 1 hour at room temperature. Microplate wells were then washed 5 times as previously, then 100 μl CDP-star substrate was added to wells and incubated with mild shaking for 5 min at room temperature. At the end of the incubation period, reactions were transferred to glass tubes containing 400 μl of CDP-star assay buffer (Tropix) and ensuing chemiluminescence was measured in an AutoLumat LB953. Representative results of this procedure are depicted in FIG. 2 and FIG. 3.

Materials
anti-iC3b antibody (Quidel)
EAA tube 3 with E5 IgM (Spectral Diagnostics Inc.)
LPSMax tube with LPS (Spectral Diagnostics Inc.)
Anti-iC3b Ab coated microtitre plates (Quidel)
anti-C3b alpha Antibody (Research Diagnostics)
anti-mouse IgM, alkaline phosphatase labeled (Zymed, Sigma)
CDP-star and CSPD substrate (Tropix)
Purified casein (I-Block™ regent) (Tropix)
CDP-star assay buffer (Tropix)
fresh whole blood/plasma
Tween-20 (Sigma)
Sodium azide (Sigma)
appropriate buffers:
   0.1 M borate buffer pH 9.5
   TBS pH 7.4 and pH 7.6
   TBS-T pH 7.6
   CA buffer pH 7.6
   ELISA-Light blocking buffer (Tropix)
Pyroplates (Cape Cod Inc)
   Equipment: shakers, incubators end-over-end rotors, Berthold AutoLumat chemiluminometer
      Results

Initial assay using anti-iC3b antibody coated beads. Results for initial testing of bead conjugates with LPS and E5 IgM are depicted in FIG. 1. Signal to noise ratio is higher using EDTA plasma than with whole blood due to the well-known non-specific chemiluminescence phenomenon of haeme. The graph also indicates that maximal chemiluminescence is achieved by 5-10 min suggesting an ideal time for chemiluminescence assessment in end-point assays. While chemiluminescence is higher in the presence of LPS than in the absence of it, this assay condition is suboptimal as there is considerable signal in the absence of LPS (negative control). Further optimizations are required to establish optimal incubation times, antibody concentrations, bead conjugate concentrations, sample amount, secondary antibody make, and diluents in order to lower non-specific signal.

Early optimization results using anti-iC3b antibody coated microtitre plates. FIG. 2 indicates that under current conditions, anti-IgM-ALP (Zymed) dilutions between 1 in 10,000 and 1 in 50,000 give sufficient signal after 5 minutes of incubation. Therefore, for subsequent experiments, this antibody was used at 1 in 20,000 or 1 in 25,000 dilutions. It should be noted that use of this secondary antibody obtained from other manufacturers or change in the amount of other reagents of the assay may require re-establishment of the optimal dilution. FIG. 3 depicts how sensitivity of the assay and dose response to LPS can change with variations in the amount of E5 IgM added to the sample.

What is claimed is:

1. An assay useful to determine the presence or amount of an analyte present in a sample of body fluid, the assay comprising:
treating the sample to form, in vitro, a reagent complex comprising analyte present in the sample, non-human antibody to the analyte, and a fixed complement component; and
reacting the reagent complex with a labeled ligand for said non-human antibody to the analyte,
whereby the presence or quantity of detectable label indicates the presence or quantity of analyte in the sample.

2. The assay according to claim 1, wherein the reagent complex is captured by an immobilized ligand for the complement component of the reagent complex.

3. The assay according to claim 2, wherein the immobilized ligand is a ligand immobilized by conjugation to a magnetic bead.

4. The assay according to claim 1, wherein said label is alkaline phosphatase.

5. The assay according to claim 4, wherein said label is detected by measuring chemiluminescence generated by reaction products of an alkaline phosphatase substrate.

6. The assay according to claim 1, wherein the analyte is LPS.

7. The assay according to claim 2, wherein the non-human antibody is an IgM.

8. The assay according to claim 7, wherein the complement fixed by the reagent complex is iC3b.

9. The assay according to claim 2, wherein the immobilized ligand is anti-iC3b antibody.

10. The assay according to claim 9, wherein the immobilized ligand is anti-iC3b antibody conjugated to a magnetic bead.

11. An assay useful to determine the presence or amount of endotoxin present in a sample of plasma, the assay comprising treating the sample to form, in vitro, a reagent complex comprising endotoxin present in the sample, non-human antibody to endotoxin, and complement fixed by the immune complex formed between said endotoxin and non-human antibody; and
reacting the reagent complex with a labeled ligand for said non-human antibody, whereby the presence or quantity of detectable label indicates the presence or quantity of endotoxin in the sample.

12. The assay according to claim 11, wherein the reagent complex is captured by an immobilized ligand for the complement component of the reagent complex.

13. The assay according to claim 12, wherein the immobilized ligand is a ligand immobilized by conjugation to a magnetic bead.

14. The assay according to claim 11, wherein said label is alkaline phosphatase.

15. The assay according to claim 14, wherein said label is detected by measuring chemiluminescence generated by reaction products of an alkaline phosphatase substrate.

16. The assay according to claim 12, wherein the non-human antibody is an IgM.

17. The assay according to claim 16, wherein the non-human antibody is E5.

18. The assay according to claim 17, wherein the complement fixed by the reagent complex is C3b.

19. The assay according to claim 2, wherein the immobilized ligand is anti-C3b antibody.

20. The assay according to claim 9, wherein the immobilized ligand is anti-iC3b antibody conjugated to a magnetic bead.

21. A kit useful to perform an assay for determining the presence or amount of analyte present in a sample of body fluid, the kit comprising a non-human antibody to said analyte, a labeled antibody for said non-human analyte, and instructions for the use thereof in accordance with the assay defined in claim 1.

22. The kit according to claim 21, further comprising a solid phase having immobilized thereon a ligand for binding a reagent complex comprising said non-human antibody, said analyte, and a complement component fixed thereto.

23. The kit according to claim 21, wherein said non-human antibody binds endotoxin.

24. The kit according to claim 21, wherein said labeled antibody comprises an alkaline phosphatase label.
